(19) United States
(12) Plant Patent Application Publication
(10) Pub. No.: US 2002/0073469 P1
(43) Pub. Date: Jun. 13, 2002

(54) Variety of geranium plant named 'Penorg'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,158

(22) Filed: Dec. 8, 2000

(51) Int. Cl. A01H 5/00

(52) U.S. Cl. PLT/330

(57) ABSTRACT

A new and distinct zonal geranium plant with red colored flowers above medium green foliage. The plant is suitable for planting in big pots.

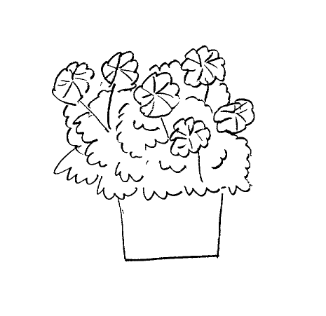

### BACKGROUND OF THE INVENTION

The present invention comprises a new and distinct cultivar of Pelargonium x hortorum known by the varietal name ‘Penorg’. The new variety was discovered in a selective breeding program in Dresden, Germany. The new variety is the result of cross breeding of a group of about 50 seedlings having, semi-double and double red-orange colored flowers. The new variety was first asexually reproduced by cuttings in Dresden, Germany. The new variety has been trial and field tested and has been found to retain its distinctive characteristics and remain true to type through successive propagations.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to The R.H.S. Colour Chart of The Royal Horticultural Society of London.

Plant:

Classification.—Botanical: Pelargonium x hortorum. Commercial: Zonal geranium.

Form.—Erect.

Height from media surface to top of foliage.—25 cm.

Height from media surface to top of umbel.—40-50 cm.

Growth.—Erect, medium degree of branching.

Strength.—Does not require artificial support.

Response time.—3.5 weeks.

Leaves.—Size: 7-8 cm long; 7-9 cm wide. Shape: Cordate. Margin: Emarginated; ondulated. Texture: Pubescent. Color: Upper surface: Green Group 137B. Lower surface: Green Group 138A. Ribs and veins: Color: Brighter than leaf surface.

Petioles.—Color: Green Group 128A. Length: 6-7 cm.

Stem/branches.—Color: Green Group 138A. Internode length: 3-4 cm. Lateral branch length: 20-25 cm.

The bud:

Shape when just showing color.—Overall: Spherical. Individual bud: Elliptical.

Color.—Green Group 138A.

Size when just showing color.—Umbel: 10-12 cm. across. Individual bud: 1.0-1.5 cm. long; 0.5-0.7 cm wide.

Inflorescence:

Blooming habit.—Semi-double.

Borne.—Flower on umbel; umbel on pedicel; pedicel on peduncle. 30-40 flowers per umbel.

Umbel.—10-12 cm. across; 8-10 cm. deep.

Flowers.—Size: 4-5 cm. across; 1-5 cm. deep. Form: Zygomorph. Number of petals: 12-15. Petals: Size: 2-3 cm long; 2 cm long. Color: Upper surface: Red Group 40A. Lower surface: Red Group 41B. Shape: Overall: Round—elliptic. Apex: Round. Base: Narrow. Margin: Smooth. Texture and appearance: Velvety.

Sepals.—Color: Upper surface: Green Group 143C. Lower surface: Yellow-Green Group 144A. Shape: Overall: Elliptic. Apex: Pointal. Base: Blunt. Size: 0.8-1.0 cm. long; 0.3-0.4 cm. wide.

Petaloids.—Quantity: 4-5. Shape: Flakes. Color: Red Group 40A.

Pedicel.—Length: 2.5-3 cm. Color: Yellow-Green Group 146A.

Peduncle.—Length: 10-12 cm. Color: Yellow-Green Group 144A.

Disease/pest resistance.—No unusual susceptibility to diseases or pests has been noted to date.

Natural flowering season.—summer, frost free period.

Lasting quality.—Long-lasting.

Reproductive organs:

Stamens.—Anthers: 3 mm long. Filaments: Length: 0.5 cm maximum. Color: Pink. Pollen: Yellow, medium amount.

Pistils.—Number: Five. Length: 0.3-0.4 cm. Stigma: Pink. Style: 0.8 cm long. Color: Greenish.

Ovaries.—Greenish, superior, 0.4 cm long.

Fruit.—Some present. Size: 0.5×0.2 cm. Color: Brown

### GENERAL CHARACTERISTICS

1. Compact.

2. High ornamental value.

3. Shiny poppy red.

4. Suitable for big pots.

I claim:

1. A new and distinct variety of geranium plant substantially as shown and described.
